(19) United States
(12) Patent Application Publication
(10) Pub. No.: US 2010/0326484 A1
(43) Pub. Date: Dec. 30, 2010

(54) VIBRATING AND ULTRASONIC WASHER

(76) Inventor: CHIH-HSIEN WU, Taipei (TW)

Correspondence Address: LEONG C. LEI, PMB # 1008, 1839 YGNACIO VALLEY ROAD, WALNUT CREEK, CA, 94598, US

(21) Appl. No.: 12/628,159

(22) Filed: Nov. 30, 2009

(30) Foreign Application Priority Data
Jun. 25, 2009 (TW) 098211499

(51) Int. Cl. A61L 2/24; A61L 2/10; A61L 2/025; B08B 3/12

(52) U.S. Cl. 134/56 R; 134/140; 134/115 R; 134/113

(57) ABSTRACT

A vibrating and ultrasonic washer which is composed of a case externally provided with a cover and a switch button, and interiorly provided with control circuits, a UV sterilizing light and a vibration device; wherein the vibration device includes pluralities of support mats, a vibration motor and/or at least an ultrasound generator and a washing tank; the vibration motor and/or at least one ultrasound generator is/are provided on a base board and controlled by the control circuits; the base board is connected into the case via the pluralities of support mats; the washing tank is stacked on the base board; thereby the articles to be washed can be put in the washing tank to accomplish cleaning and sterilizing purposes via the vibration waves and/or ultrasound.

### FIELD OF THE INVENTION

The present invention relates to a vibrating and ultrasonic washer, particularly to a vibrating washer which has vibration waves and/or ultrasound, and has a UV light for the purpose of effectively cleaning and sterilizing.

### BACKGROUND OF THE INVENTION

While daily lives start from brushing teeth and washing face, the toothbrush generally would become humidified after use, and the filth from the teeth would easily remain in the toothbrush, rendering the toothbrush full of germs. In this case, the germs will enter the user's body through brushing teeth next time; therefore, cleaning toothbrush is an extremely important thing in daily lives. Likewise, dentures, orthopedic tooth brackets or sports protective tooth mount cannot be thoroughly cleaned. Generally, people would put their dentures or tooth mounts in the water or mouthwash first, and brush them manually, which is time-consuming but to poor avail, and even occurs scratch on the dentures or tooth mount. Time after time, dental plaque would easily occur on the dentures or tooth mount, and multiplied between teeth and on the surface of dentures and tooth mount. Accordingly, efficiently cleaning the teeth and tooth-related products is of extreme importance.

In addition, grime or dust would accumulate in jewelry such as rings, necklaces, brooches, etc. after being worn for a period of time. It is necessary to bring the jewelry to a special shop for cleaning purpose. If, however, there is an easily operated machine serviceable for such a cleaning work at home, people can wash their jewelry themselves.

Furthermore, the number of people who get myopia is increasing nowadays, and contact lenses, which can rectify eyesight and make a wearer look like a normal people, are daily articles for those myopic people. However, wearing contact lenses would occur accumulation of protein precipitate and make the wearer feel uncomfortable due to bad gas permeability of the contact lenses. Therefore, the contact lenses need to be cleaned after use everyday, and if the detergent solution can be vibrated during cleaning process, the contact lenses can be thoroughly cleaned to prevent the wearer's eyes from bacterial infection or keratitis, as well as to prolong the life of use of the contact lenses.

In view of the above, the invention relates to an effective and convenient washer, which is subject to the needs of washing toothbrush head, denture, tooth bracket, jewelry and contact lenses constantly, with an application of a vibrating and ultrasonic washer, and accompanied with the function of a UV sterilizing light for cleaning articles to be washed.

### SUMMARY OF THE INVENTION

The primary object of the invention is to provide a vibrating and ultrasonic washer, which is simply structured, delicately sized, and portable as desire, as well as easy to accomplish a cleaning purpose via vibrating and ultrasonic ways.

Another object of the invention is to provide a vibrating and ultrasonic washer, which is provided with a UV light for sterilizing and disinfecting purposes.

To obtain the above objections, the vibrating and ultrasonic washer of the invention is composed of a case which is externally provided with a cover and a switch button, and interiorly provided with control circuits and a vibration device. The vibration device includes pluralities of support mats, a vibration motor, at least an ultrasound generator and a washing tank. The vibration motor and the ultrasound generator are provided on a base board and controlled by the control circuits. The base board is connected into the case via the pluralities of support mats, whereas the washing tank is attacked on the base board. Thereby, the article to be washed can be put in the washing tank, and be completed cleaned via the vibration of the vibration motor and the ultrasound.

In the above-mentioned vibrating and ultrasonic washer, the cover is pivotally provided with a transparent lid.

In the vibrating and ultrasonic washer, the pluralities of support mats are made of shock-absorbent and sound-absorbent silicone.

In the vibrating and ultrasonic washer, the case is interiorly provided with a battery case and an alternating current (AC) socket for supplying power to the control circuits.

In the vibrating and ultrasonic washer, the case is provided on its top with a UV sterilizing light; meanwhile a safety design of magnetic spring switch is provided for automatically shut the UV light as soon as the cover is opened.

In the vibrating and ultrasonic washer, the vibrating device is provided with two ultrasound generators, which are combined to the two sides of the vibration motor.

Accordingly, the invention has the following advantages:

1. It is extremely convenience to use the vibrating and ultrasonic washer which is simply structured, delicately sized, and applicable to both AC and DC power.

2. The provision of the cover can ease the user to clean the washing tank separately; meanwhile, the opening of the transparent lid of the cover may allow the user to insert the head of a toothbrush conveniently.

3. After a easy wash of dentures, tooth brackets, jewelry or contact lenses, the user only needs to cover up the cover of the washing tank and proceed with vibrating and ultrasonic washing to obtain a completely clean purpose, time and effort saving.

4. As the vibration motor, ultrasound generator(s) and case of the vibrating and ultrasonic washer are combined via support mats made of silicone, the vibration and noise occurred by the motor and ultrasound generator(s) can be absorbed/muffled via the silicone.

To completely appreciate these and other objects of the present invention as well as the invention itself, all of which will become apparent to those skilled in the art, the following detailed description of the invention and the claims should be read in conjunction with the accompanying drawings.

### BRIEF DESCRIPTION OF THE DRAWINGS

FIG. 1 is a perspective view of the present invention.

FIG. 2 is an exploded view of the present invention.

FIG. 3 is an exploded view of the cover.

FIG. 4 is an exploded view of the vibration device.

FIG. 5 is a cross-sectional view of the invention.

FIG. 6 is a perspective view showing separation of the washing tank from the case.

FIG. 7 is a cut-away view of the invention showing the use status thereof.

FIG. 8 is a perspective view of the invention showing the use status thereof.

### DETAILED DESCRIPTION OF THE PREFERRED EMBODIMENTS

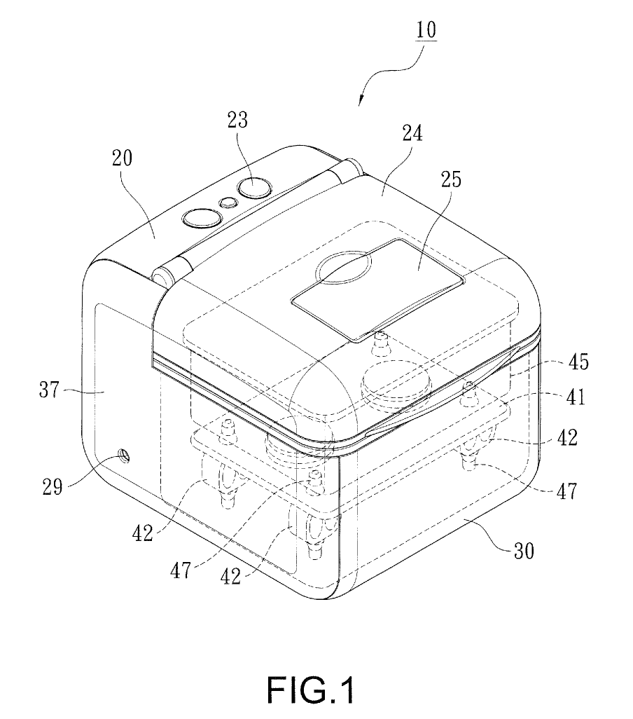

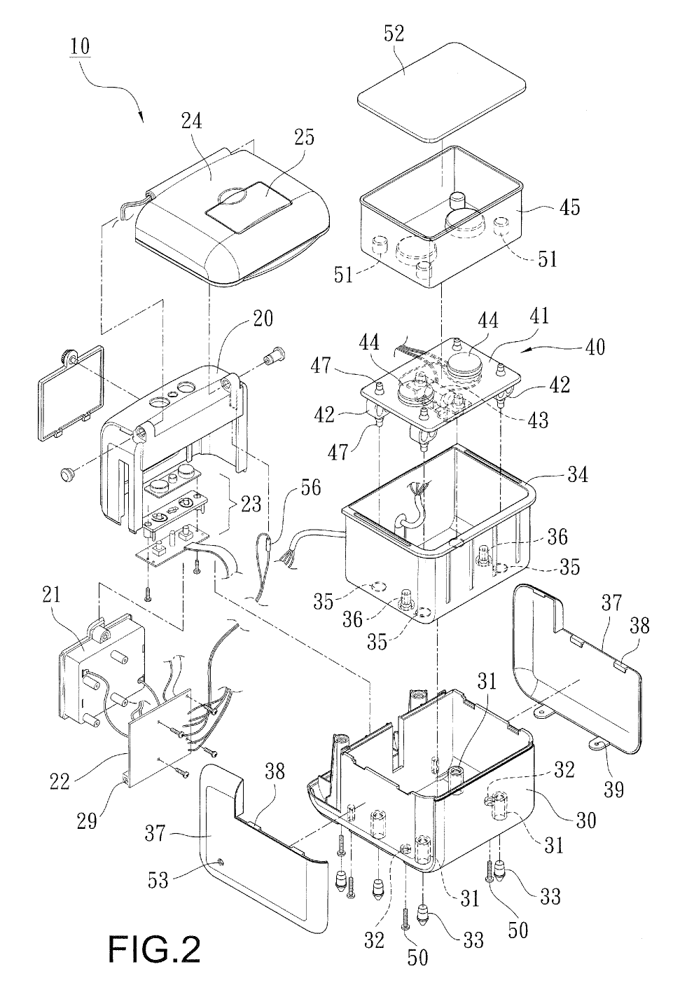

Referring to FIGS. 1 and 2, the vibrating and ultrasonic washer 10 of the intention is provided with a front cover 20, a lower case 30 and a vibration device 40. Under the front cover 20, a battery case 21 is screwed to the front cover 20 and connected with a circuit board 22 provided with control circuits. The circuit board 22 is connected to the switch buttons 23 on the top of the front cover 20. The switch buttons 23 expose out of the top of the front cover 20, including those for vibration motor 43, UV light 28 and ultrasound generator(s) 44. The four corners inside the lower case 30 are provided with lockholes 32 and hollow posts 31 with soft pad feet 33 mounted on the bottom thereof. The lower case 30 is interiorly provided with a inner case 34, on the four corners of which are provided with holes 35 and connection holes 36 corresponding to the hollow posts 31 and lockholes 32, respectively. Furthermore, the lower case 30 has a side board 37 at each of its right and left sides. Each side board 37 has flap hooks 38 on its upper rim, and lockholes 39 on its lower rim such that the flap hooks 38 can lock on the upper rim of the two sides of the lower case 38, that the lockholes 39 can combine with the lockholes 32 on the bottom of the lower case 30 and that lock holes 39 & 32 can be combined with connection holes 36 via screws 50, thereby the side boards 37, lower case 30 and inner case 34 can be combined together. The circuit boards 22 is provided with a power socket 29, whereas the corresponding position on the side board 37 is provided with an aperture 53 for connecting AC that is converted by a transformer.

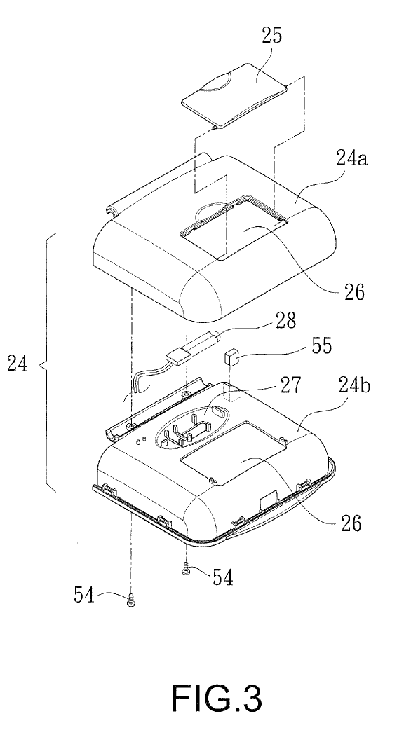

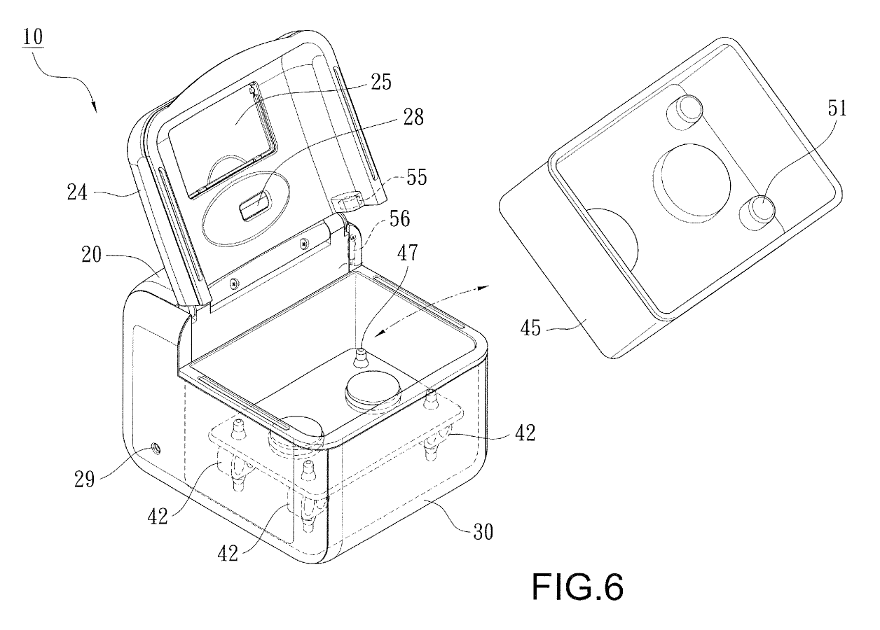

Now referring to FIG. 3, the top of the front cover 20 is further pivotally provided with a cover 24 which is composed of upper housing 24a and lower housing 24b of substantially the same shape, each of which is provided with a opening 26 corresponding to each other. A transparent lid 25 is pivotally connected to the cover 24 to form an open gate. The lower housing 24b is provided with an indentation 27 aside the opening 26 for combining with a UV light 28. By way of screws 54, the upper housing 24a and lower housing 24b can be fastened together. There is a magnet 55 aside the UV light 28, whereas a magnetic spring switch 56 is provided on the front cover 20 (see FIGS. 2 and 6) such that when the cover 24 is opened, the UV light 28 would automatically be turned off to prevent the UV light 28 from irradiating the user's eyes.

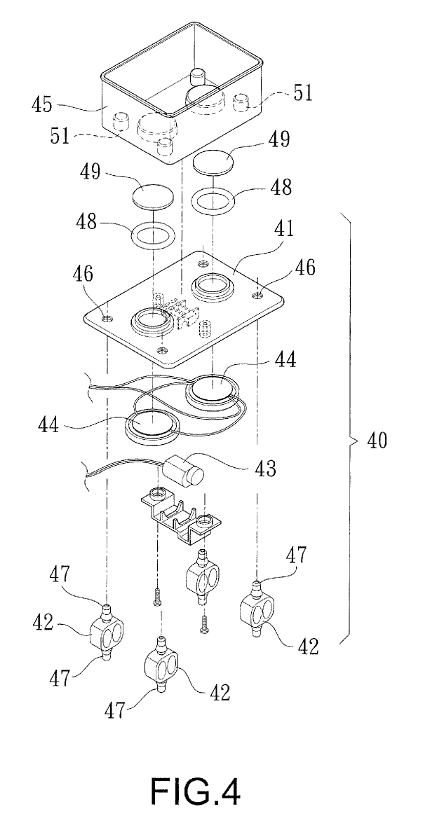

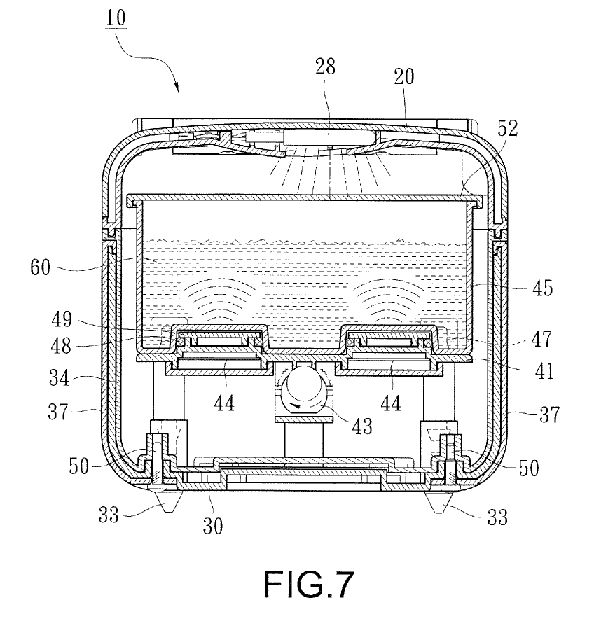

Referring to FIG. 4, the vibration device 40 is provided with a base board 41, four support mats 42, a vibration motor 43, two ultrasound generators 44 and a washing tank 45. Connecting holes 46 are provided on the corresponding positions on the base board 41; the top and bottom of the four support mats 42 are provided with silicone pads 47 which can go through the connecting holes 46 for the purposes of engagement and padding under the washing tank 45. Anti-leakage rings 48 and pressure plates 49 are provided between the vibrating device 40 and the washing tank 45. The vibration motor 43 is connected to the central bottom of the base board 41; the two ultrasound generators 44 are also connected to the bottom of the base board 41 but distributed at the two sides of the vibration motor 43. The four support mats 42 are made of shock-absorbent and sound-absorbent silicone rubber for engagement of the base board 41 in the inner case 34. Furthermore, a socket 51 is provided at each bottom corner of the washing tank 45. The sockets 51 are provided in coordination with the positions of the extruding silicone pads 47, thereby engaging the washing tank 45 into the inner case 34. When the washing tank 45 is installed in the inner case 34 in a manner that the outer wall of the washing tank 45 is 4 mm distant from the interior wall of the inner case 34 such that when the washing tank 45 would not touch the inner case 34 when being shaken by the vibration motor 43. Meanwhile, a cover 52 can be provided on the top of the washing tank 45 to prevent liquid 60 from splashing out of the washing tank 45 (see FIG. 7).

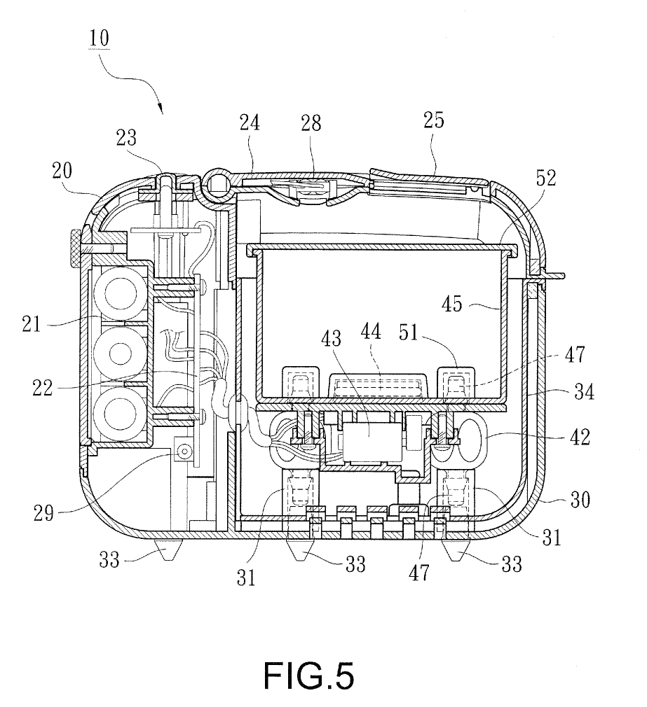

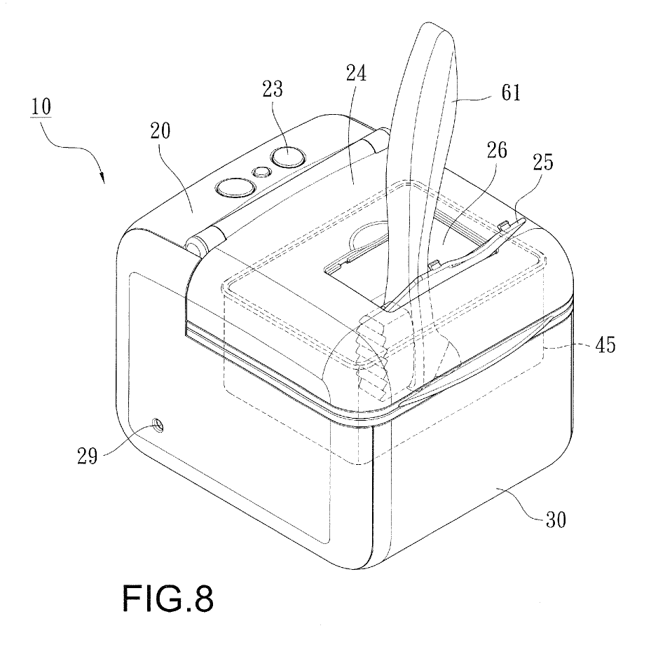

As shown in FIGS. 5-8, the control circuits of the circuit board 22 connect with the UV light 28, magnetic spring switch 56, vibration motor 43 and two ultrasound generators 44. As shown in FIG. 7, when the washing tank 45 is filled with liquid 60 such as water or sterilization liquid, the user may press the switch button 23 to start the vibrating and ultrasonic washer 10. The vibration motor 43 and the two ultrasound generators 44 can be started individually or concomitantly to generate vibration and/or ultrasonic. The UV light 28 can alternatively be controlled by the switch buttons 23 to start the UV ray sterilizing function. After the transparent lid 25 is opened, the user can put the head of a toothbrush 61 into the washing tank 45 (as shown in FIG. 8) for washing in the water via vibrating wave and/or ultrasound, as well as for sterilizing via UV rays, thereby accomplishing a thorough cleaning purpose. After the toothbrush 61 has been completely cleaned, the user needs only to open the cover of the vibrating and ultrasonic washer 10 and take out the washing tank 45 alone for cleaning purpose.

When the vibrating and ultrasonic washer 10 is used for cleaning a toothbrush 61, the user needs to take away the cover 52 of the washing tank 45, and directly put the head of the toothbrush 61 into the washing tank 45. However, when the articles to be washed refer to dentures, orthopedic tooth brackets, sports mouthpieces, jewelry or contact lenses, after the detergent liquid 60 (or caring liquid) is filled into the washing tank 45, the cover 52 should be shut to prevent the liquid 60 from splashing out.

Concluded above, the vibrating and ultrasonic washer 10 according to the invention can accomplish cleaning work via two different modes, and has the function of UV light sterilization.

While certain novel features of this invention have been shown and described and are pointed out in the annexed Claims, it is not intended to be limited to the details above, since it will be understood that various omissions, modifications, substitutions and changes in the forms and details of the device illustrated and in its operation can be made by those skilled in the art without departing in any way from the spirit of the present invention.

1. A vibrating and ultrasonic washer, composed of a case which is externally provided with a cover and a switch button, and interiorly provided with control circuits and a vibration device; the vibration device includes pluralities of support mats, a vibration motor and a washing tank; the vibration motor is provided on a base board and controlled by the control circuits; the base board is connected into the case via the pluralities of support mats.

2. The vibrating and ultrasonic washer according to claim 1, wherein a transparent lid is pivotally provided on the cover.

3. The vibrating and ultrasonic washer according to claim 1, wherein the case has a battery case and a power socket.

4. The vibrating and ultrasonic washer according to claim 1, wherein the pluralities of support mats are made of shock-absorbent and sound-absorbent materials.

5. The vibrating and ultrasonic washer according to claim 1, wherein the top of the case is provided with a sterilizing ultraviolet (UV) light.

6. The vibrating and ultrasonic washer according to claim 5, wherein a magnet is provided aside the UV light, and a magnetic spring switch is provided above the front cover.

7. A vibrating and ultrasonic washer, composed of a case which is externally provided with a cover and a switch button, and interiorly provided with control circuits and a vibration device; the vibration device includes pluralities of support mats, at least an ultrasound generator and a washing tank; the ultrasound generator is provided on a base board and controlled by the control circuits; the base board is connected into the case via the pluralities of support mats, and the washing tank is stacked on the base board.

8. The vibrating and ultrasonic washer according to claim 7, wherein the cover is pivotally provided with a transparent lid.

9. The vibrating and ultrasonic washer according to claim 7, wherein the case has a battery case and a power socket.

10. The vibrating and ultrasonic washer according to claim 7, wherein the pluralities of sport mats are made of shock-absorbent and sound-absorbent materials.

11. The vibrating and ultrasonic washer according to claim 7, wherein the top of the case is provided with a sterilizing ultraviolet (UV) light.

12. The vibrating and ultrasonic washer according to claim 11, wherein a magnet is provided aside the UV light, and a magnetic spring switch is provided above the front cover.

13. A vibrating and ultrasonic washer, composed of a case which is externally provided with a cover and a switch button, and interiorly provided with control circuits and a vibration device; the vibration device includes pluralities of support mats, a vibration motor, at least an ultrasound generator and a washing tank; the vibration motor and the ultrasound generator are provided on a base board and controlled by the control circuits; the base board is connected into the case via the pluralities of support mats, and the washing tank is stacked on the base board.

14. The vibrating and ultrasonic washer according to claim 13, wherein the cover is pivotally provided with a transparent lid.

15. The vibrating and ultrasonic washer according to claim 13, wherein the case has a battery case and a power socket.

16. The vibrating and ultrasonic washer according to claim 13, wherein the pluralities of sport mats are made of shock-absorbent and sound-absorbent materials.

17. The vibrating and ultrasonic washer according to claim 13, wherein the top of the case is provided with a sterilizing ultraviolet (UV) light.

18. The vibrating and ultrasonic washer according to claim 17, wherein a magnet is provided aside the UV light, and a magnetic spring switch is provided above the front cover.

19. The vibrating and ultrasonic washer according to claim 13, wherein the vibration device is provided with two ultrasound generators; the vibration motor is combined to the center of the base board, and the two ultrasound generators are combined to the two sides of the vibration motor.
